(19) United States
(12) Patent Application Publication
(10) Pub. No.: US 2005/0273172 A1
(43) Pub. Date: Dec. 8, 2005

(54) Artificial disc and uses therefor

(76) Inventors: Arun Angelo Patil, Omaha, NE (US); L. Russell Alberts, Omaha, NE (US)

Correspondence Address: PATTERSON & SHERIDAN, L.L.P., 3040 POST OAK BOULEVARD, SUITE 1500, HOUSTON, TX, 77056, US

(21) Appl. No.: 10/862,529

(22) Filed: Jun. 7, 2004

(51) Int. Cl. A61F 2/44

(52) U.S. Cl. 623/17.16

(57) ABSTRACT

Disclosed are prosthetic devices for insertion into intervertebral disc spaces after the removal of an intervertebral disc.

### FIELD OF THE INVENTION

The present invention is directed to prosthetic devices which may be inserted into intervertebral spaces resulting from the removal of diseased or damaged intervertebral discs.

### BACKGROUND OF THE INVENTION

Disc disease is a degenerative disease of the intervertebral disc characterized by chronic and disabling spine and upper extremity pain or sensory deficits. When a patient fails non-surgical treatment, discectomy is needed. Discectomy involves the removal of disc material from the space between two vertebrae, the neural foramen and spinal canal. Though discectomy relieves the pressure on the affected nerve root and spinal cord, discectomy creates a space between two vertebrae in the spinal column. To maintain the height of the space following removal of disc material, a piece of bone or a bone-filled cage is often fused into the disc space. One of the major disadvantages of fusion, however, is the loss of movement between the fused vertebral segments, which in turn leads to increased mobility in the spinal segments above and below the fusion predisposing them to degeneration, and, possibly, herniation. To obviate this problem, artificial discs have been designed.

One of the early designs of artificial discs is shown in U.S. Pat. No. 4,309,777, and consists of a spring-loaded core and two end plates with spikes. Another type of artificial disc consists of a rubber core vulcanized to two titanium end plates that have posts to provide initial fixation and porous in-growth surfaces for long-term fixation (see, e.g., Enker, et al., Spine 18:1061-70 (1993); and Wigfield, et al., Spine 27(22): 2446-52 (2002)). Another type of artificial disc known in the art consists of a polyethylene core and two cobalt-chromium alloy end plates with spikes (see, e.g., Griffith, et al., Spine 19:1842-49 (1994); and Zeegers, et al., Eur. Spine 8:210-17 (1999)). All of the aforementioned discs have flat metal surfaces with spikes or posts to anchor and maintain the position of the disc in the disc space. The problems encountered by these discs are breakage or separation of the disc components and slippage of the implants. Another prior art device consists of a hydrogel core that is encased in a polyethylene jacket and is intended to preserve disc height while permitting normal range of motion (Klara and Ray, Spine 27(12):1374-77 (2002)); while yet another system uses bioceramic-coated three-dimensional fabric to maintain space height and allow bone in-growth to attach the artificial disc to the vertebral body (Takahata, et al., Spine 28:637-44 (2003)).

There is, however, need in the art for an inexpensive, effective artificial disc that preserves the spacing between vertebrae, while providing adequate support without undue compression or slippage long term. The present intervertebral device serves this need.

### SUMMARY OF THE INVENTION

The present invention, in certain embodiments, is directed to prosthetic intervertebral devices designed to accommodate the specific morphological anatomy of vertebral endplates. Once inserted into the intervertebral disc space, the inventive devices aid in reconstruction of the disc space resulting from the removal of a single damaged or diseased disc or multiple damaged or diseased discs from any location in the spine.

Thus, in one embodiment, the present invention provides a prosthetic intervertebral device consisting essentially of a unitary body comprising an elastomer, such as a silicone elastomer or a silicone-like elastomer. The prosthetic intervertebral devices according to the present invention may have any appropriate shape, including those that are rectangular (i.e., a three-dimensional rectangular body), rectangular with one or more recessed or rounded edges, disc- or “hockey puck”-shaped (oval), or contoured. Preferably the prosthetic intervertebral device consists essentially of an elastomer, such as a silicone-containing elastomer or a silicone-like elastomer, more preferably the prosthetic intervertebral device consists essentially of medical grade silicone, having a durometer (hardness) of about 10 D to about 400 D, and even more preferably about 20 D to about 300 D or 25 D to about 200 D.

In yet another embodiment, the present invention provides a method for preserving intervertebral disc space after discectomy comprising implanting the intervertebral device of the present invention into the intervertebral disc space.

These and other objects, advantages and features of the present invention will become apparent to those persons skilled in the art upon reading the details of the structure of the devices and methods of use, as set forth below more fully.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIG. 1 is a left side elevation view of the spinal column (A) showing the cervical spinal region (A1), the thoracic spinal region (A2), the lumbar spinal region (A3), and the sacral spinal region (A4).

FIG. 2 is a detailed perspective view of FIG. 1 showing the front and left sides of lumbar vertebrae (the A3 region of FIG. 1) as well as one embodiment of the inventive intervertebral device (D).

FIG. 3A is a side elevation view of one embodiment of the inventive device positioned on the superior surface of a contoured inferior vertebral endplate. FIG. 3B is a front elevation view illustrating an embodiment of the inventive device positioned on the superior surface of the contoured inferior vertebral endplate.

FIG. 4A is a right side elevation view of one embodiment of the inventive device positioned on inferior surface of a superior vertebral endplate. FIG. 4B is a front elevation view of one embodiment of the inventive device positioned on the inferior surface of a superior vertebral endplate.

FIG. 5A is a side elevational view of a rectangular embodiment of an intervertebral device according to the present invention with exemplary measurements shown. FIG. 5B is a side-sectional view of the intervertebral device shown in FIG. 5A.

FIG. 6A is a side elevational view of a recessed-edge rectangular embodiment of an intervertebral device according to the present invention with exemplary measurements shown. FIG. 6B is a side-sectional view of the intervertebral device shown in FIG. 6A.

FIG. 7A is a side elevational view of a contoured embodiment of an intervertebral device according to the present invention. FIG. 7B is a side-sectional view of the intervertebral device shown in FIG. 7A.

FIG. 8A is a side elevational view of a disc-shaped or puck-shaped embodiment of an intervertebral device according to the present invention. FIG. 8B is a side-sectional view of the intervertebral device shown in FIG. 8A.

FIG. 9A is a side-sectional view of the contoured embodiment of the artificial disc shown in FIGS. 7 and 7B. The sites of thickness measurements are shown. FIG. 9B is a chart showing the average and standard deviation change in dimension for a 30 D contoured disc at one million cycles. FIG. 9C is a chart showing the thickness change in mm for a 30 D contoured disc at five million cycles.

### DETAILED DESCRIPTION

Before devices and methods of the present invention are described, it is to be understood that this invention is not limited to the particular methodology or apparatus described, as such methods or apparatus may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and” and “the” include plural referents unless the context clearly dictates otherwise.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention. For example, additional descriptions and uses for vertebral prostheses such as those disclosed herein include those described in U.S. Pat. Nos. 6,482,234 to Weber, et al.; 6,264,695 to Stoy, et al.; 6,022,376 to Assell, et al.; 5,192,326 to Bao, et al.; 4,863,477 to Monson, et al., and 5,171,281 to Parsons, et al. all disclosing encapsulated devices; 5,545,229 to Parsons, et al. showing an encapsulated device with an endplate; 5,458,643 and 5,314,478 both to Oka, et al. showing layered devices; 6,436,146 to Hassler, et al.; 6,240,926 and 5,964,807 to Gan, et al.; 6,066,174 to Farris, et al.; 5,514,180 to Heggeness, et al.; and 5,258,043 and 5,108,438 to Stone, et al., all disclosing unitary devices; as well as US published application No. 2002/0029083 to Zuckerman showing a unitary device; and 2002/0026244 and 2003/0023311 to Trieu; 2003/0004574 to Feree; 2003/0125807 to Lambrecht, et al.; and 2003/0033017, to Lotz, et al. all disclosing encapsulated devices.

The intervertebral device of the present invention provides a number of advantages to both the surgeon and patient. The intervertebral device of the present invention is extremely easy to use: no complex tools are required to prepare an attachment area, and no complex tools or procedures are required to implant the device. In hip and knee implants, it has been shown by a number of studies that the largest variable affecting the success of the implant is not in the design of the implant or of the instruments needed to install the implant; but in the skill of the surgeon and his or her facility with the implant. A simple implant that does not require specialized preparation or installation greatly reduces the variables that arise during surgery.

Additionally, the intervertebral device of the present invention does not require attachment mechanisms that invade the vertebral bodies. By foregoing complex attachment mechanisms, the boney end plates of the vertebral bodies are preserved and risk of infection is minimized. Moreover, particles that may be generated through routine wear and tear of the intervertebral device do not have access to the marrow and cancellous bone of the vertebral bodies. The design of the present invention may be a particularly apropos option for osteoporotic patients that cannot risk having the thin cortical plates of their vertebrae compromised. Also, because the intervertebral device of the present invention is not fixed to the vertebrae, it allows for movement at the intervertebral joint.

The intervertebral device of the present invention is a unitary-component artificial disc consisting essentially of a body with a simple, straightforward design. The intervertebral device of the present invention is made of an elastomer, preferably an elastomer comprising silicone or a substance with like properties, which is soft, compressible and durable. As a spacer, the device according to the present invention maintains the height of the intervertebral space, and because the intervertebral device is soft, it grips the disc surfaces of the vertebral bodies by a clinging mechanism. This clinging mechanism is far superior to an anchoring mechanism in which an artificial disc is anchored to the vertebral bodies using spikes or screws. The superiority of the clinging mechanism is best demonstrated by the traction offered by snow tires or racing tires made of soft material. Because elastomers are compressible, the intervertebral device of the present invention contours itself to the space between the vertebrae and the surfaces of the vertebrae. Since the vertebral surfaces have a certain degree of concavity, the self-contouring property of the artificial disc of the present invention prevents it from being extruded once implanted. In addition, the axial load applied to the intervertebral device and adjacent vertebrae through the structure of the spine further increases the resistance to extrusion of the intervertebral device once implanted. The ability of the intervertebral device of the present invention to contour to the space between the vertebrae and to the vertebral surfaces thus increases as the compression increases, which is evident by the results obtained in experiments performed. The results (reported infra) show that the artificial discs with a lower durometer (hardness) are more likely to stay in position. Moreover, it was shown by experimentation (again, see infra) that reducing the durometer of the elastomer minimized the weight loss of the artificial disc over time; probably because at lower durometer, the disc conforms to the shape of the vertebral surfaces reducing the friction between the disc and the vertebral bodies.

Since the intervertebral device of the present invention is a single unit, it is less likely to break or malfunction than discs that have components. The dimensions of the intervertebral devices of the present invention can vary depending on the normal disc dimensions of the disc in the spine that is being replaced, for maintaining the normal height between vertebrae and keeping the intervertebral foramen open. Also, the intervertebral device of the present invention is resistant to wear. In summary, the intervertebral device of the present invention is a simple type of artificial disc that is durable, stable, biocompatible, non-invasive and easy to implant.

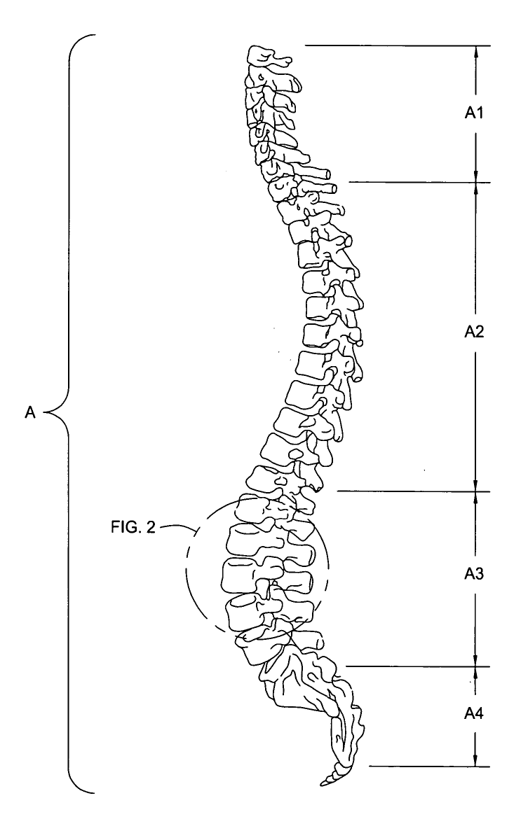

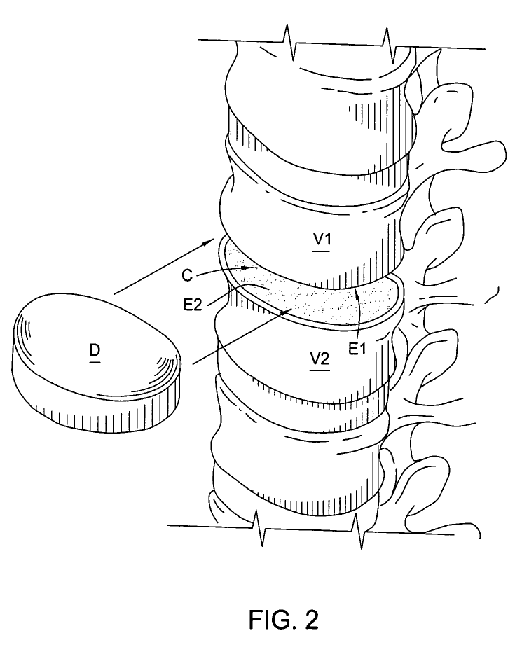

Thus, the present invention is related to prosthetic intervertebral devices intended to replace an intervertebral disc which has been removed due to disease, infection, deformity, or fracture, for example. FIG. 1 is a left side elevation view of the spinal column (A) showing the cervical spinal region (A1), thoracic spinal region (A2), lumbar spinal region (A3), and sacral spinal region (A4). FIG. 2 shows the inventive device (D) inserted into the resulting disc space (C) located between the superior endplate (E2) of the inferior vertebra (V2) and the inferior endplate (E1) of the superior vertebra (V1). Each vertebral body has a superior endplate and an inferior endplate. Because the inventive devices are made of silicone, they accommodate the defined contours (i.e. shapes) of the superior and inferior endplates of vertebral bodies. Though FIGS. 2, 3A, 3B, 4A and 4B show the device inserted into the lumbar region of the spine, it should be understood that the intervertebral device of the present invention can be used in any region of the spine; that is, the intervertebral device of the present invention may be used at one or more of the cervical spinal region, the thoracic spinal region, the lumbar spinal region and the sacral spinal region.

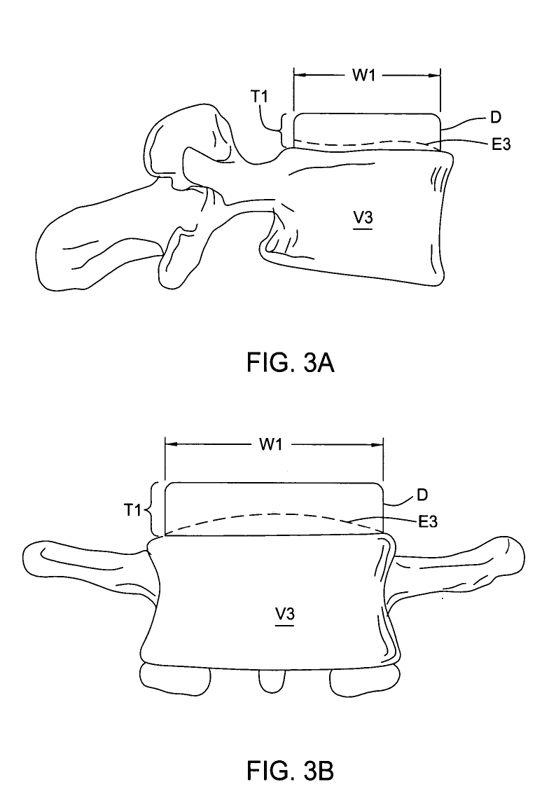

FIG. 3A is a side elevation view of one embodiment of the inventive device (D) positioned on the surface of and molded to a contoured superior endplate (E3, shown in phantom) of an inferior vertebra (V3). FIG. 3B is a front elevation view of the same vertebra (V3), illustrating the inventive device positioned on the surface (E3, also in phantom) of the contoured vertebral endplate.

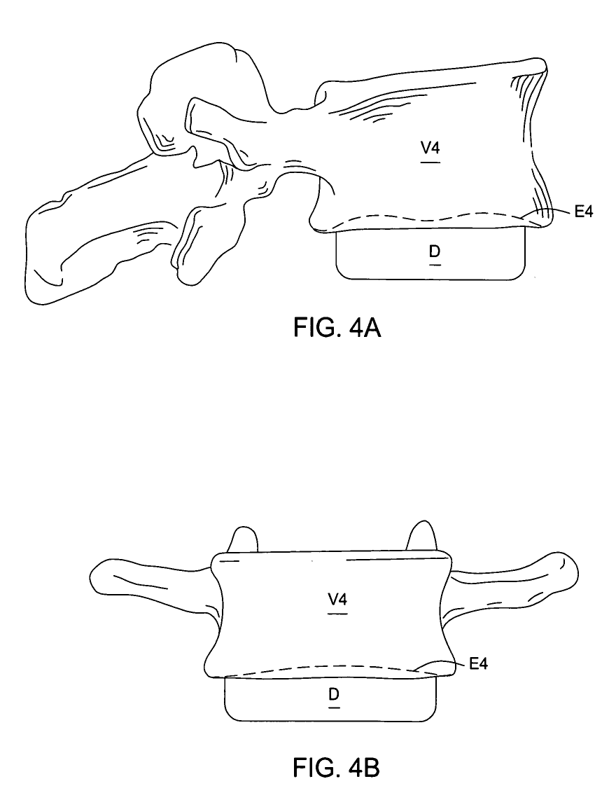

FIG. 4A is a side elevation view of one embodiment of the inventive device (D) positioned on the surface of and molded to a contoured inferior endplate (E4, shown in phantom) of a superior vertebra (V4). FIG. 4B is a front elevation view of the same vertebra (V4), illustrating the inventive device positioned on the surface (E4, also in phantom) of the contoured vertebral endplate.

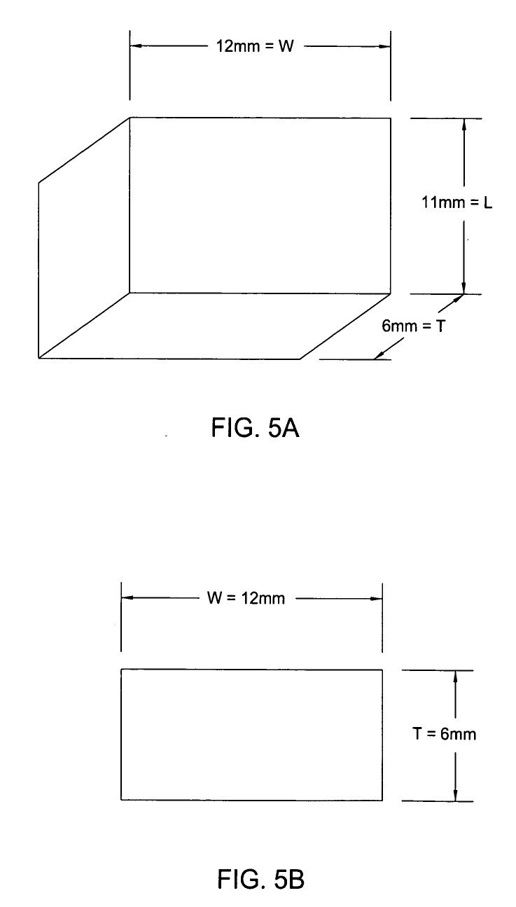

FIGS. 5A and 5B, 6A and 6B, 7A and 7B and 8A and 8B all show side elevational views and side-sectional views of various embodiments of intervertebral devices according to the present invention. The embodiment of the intervertebral device shown in FIGS. 5A and 5B is three-dimensional rectangular, having an exemplary width (lateral right-to-left dimension) W of approximately, e.g., 12 mm, an exemplary length (anterior/posterior dimension) L of approximately, e.g., 11 mm, and an exemplary thickness of approximately, e.g., 6 mm. In any embodiment of the present invention, the measurement of the disc in any dimension may vary depending on, e.g., the style of the disc, the position of the disc in the spinal column that is being replaced (vertebral level), and the size (age, gender) of the individual in which the disc is being implanted. The intervertebral device of the present invention can be made in a variety of sizes and, in practice, an appropriately-dimensioned device may be selected based on a patient pre-operative scan. Generally, however, the width (lateral right-to-left dimension) of the intervertebral device will usually be about 6-65 mm, more usually will be about 8-40 mm and even more usually will be about 10-30 mm. The length (anterior/posterior dimension) will be about 8-45 mm, more usually will be about 10-35 mm, and even more usually will be about 12-25 mm. The thickness of the intervertebral device of the present invention generally will be about 2-25 mm, more usually will be about 3-20 mm, and even more usually will be about 5-15 mm. In addition, the intervertebral device of the present invention may be 1-4 mm shorter in either one or both of the length and width than a natural disc as this allows for easier insertion. Additionally, the intervertebral device of the present invention may be 1-2 mm thicker than a natural disc to compensate for load compression. Again, however, the width, length and thickness of the intervertebral devices of the present invention range widely due to the characteristics listed above.

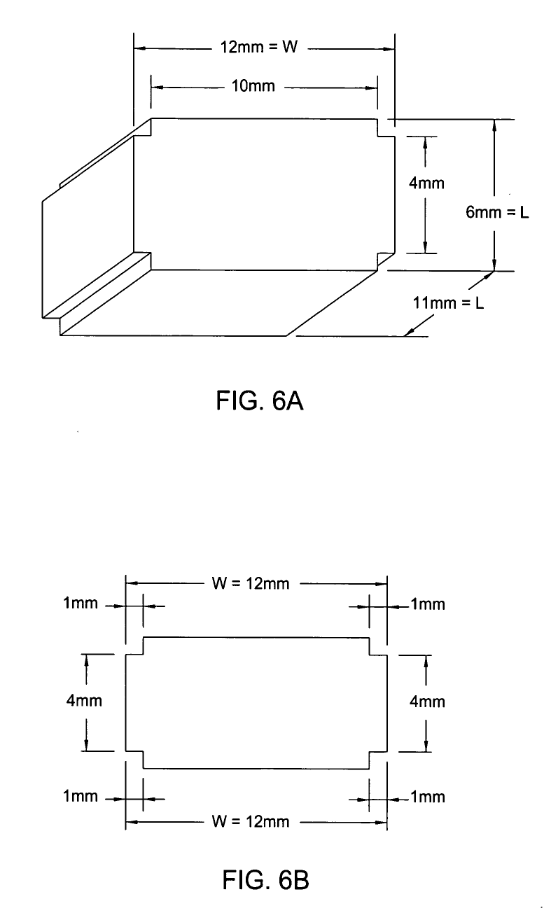

The embodiment of the intervertebral device shown in FIGS. 6A and 6B is essentially the rectangular disc shown in FIGS. 5A and 5B, having an exemplary overall width W of approximately, e.g., 12 mm, an exemplary length L of approximately, e.g., 11 mm, and an exemplary overall thickness of approximately, e.g., 6 mm. However, in this embodiment, the edges along the width on both the left and right side of the top and bottom of the disc have been recessed by, e.g., 1 mm, to give a sort of thick T-shape as shown in the side-sectional view of FIG. 6B. Alternatively, the edges may be recessed along the length of the intervertebral device or along both the width and the length of the device (embodiments not shown). Additionally, the recessed edges may be present only on the top or only on the bottom of the device, may be more or less than 1 mm, or, optimally, may be rounded rather than sharp-edged, as deemed appropriate by the surgeon.

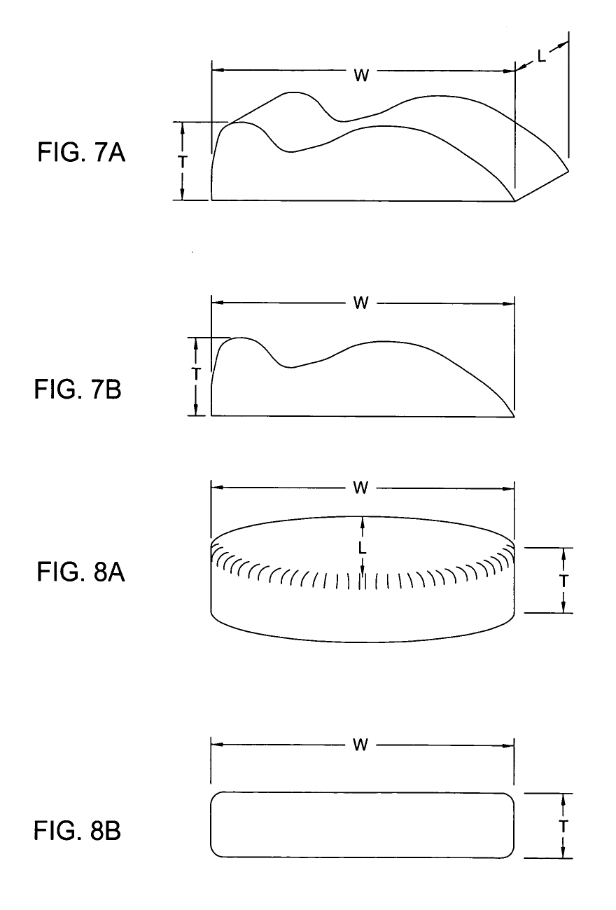

FIG. 7A is a side elevational view and FIG. 7B is a side-sectional view of one embodiment of a contoured intervertebral device. Contoured devices may be of any appropriate contour, and may be made to, for example, contour specifically to the topography of vertebrae in certain areas of the spine (e.g., the cervical spinal region, the thoracic spinal region, the lumbar spinal region, or the sacral spinal region), or accommodate certain vagaries of the adjacent vertebral endplates due, e.g., to surgery or degeneration. FIG. 8A shows a side elevational view and FIG. 8B shows a side-sectional view of a disc-shaped or “hockey puck-shaped” intervertebral device according to one embodiment of the present invention. Again, the dimensions of such an intervertebral device may vary, due to some of the factors mentioned above; however, in general the width usually will be about 6-65 mm; the length will be about 8-45 mm; and the thickness of the intervertebral device of the present invention generally will be about 2-25 mm.

The intervertebral devices of the present invention may be manufactured by any means known in the art that is appropriate for the material used in the device and that is appropriate for the manufacture of medical devices (such using techniques which maintain sterility, a high level of quality control and the like). Exemplary manufacturing processes include injection molding or hand-molding processes, e.g., such as those used by anaplastologists. Surface contours of devices, if present, may be based on the shape of cadaveric vertebral bodies, on digital imaging or other methods used to generate contours from averaging human specimens or on custom contours based on CT, MRI or other types of imaging of the patient's vertebral surfaces.

The intervertebral devices according to embodiments of the present invention comprise elastomers, such as silicone-containing or silicone-like elastomers. The elastomer that is employed may be any suitable elastomer known in the art, but includes elastomers made from medical grade silicone or other rubbers such as, e.g., 40040 or HCRA-50E, 40041 or HCRA-65E, 40042 or HCRA-80E, 40023, 40024, 40025, 40027, 40088 or HCRA-20M, 40089 or HCRA-35M, 40090 or HCRA-50M, 40082 or HCRA-80M all from Applied Silicone Corp (Ventura, Calif.); A 2186 F, 6382, 6383 all from Factor II (Lakeside, Ariz.); various formulations of silastic biomedical and medical grade high consistency rubber such as Q7-4535, Q7-4450, Q7-4565, Q7-4720, Q7-4735, Q7-4750, Q7-4765, Q7-4780, class VI elastomers such as C6-135, C6-150, C6-180, C6-350 LH Elastic, C6-355 GGR Elastic, biomedical grade liquid silicone rubber such as Silastic Q7-4840, Silastic Q7-4850, Silastic Q7-6860, Silastic 7-6830 LSR, Silastic 7-6840 LSR, medical grade low consistency rubbers such as Silastic MDX4-4210 medical grade elastomer, class VI liquid silicone rubbers such as Dow Corning C6-515 LSR, Dow Corning C6-530 LSR, Dow Corning C6-540 LSR, Dow Corning C6-550 LSR, Dow Corning C6-560 LSR, or Dow Corning C6-570 LSR all from Dow Corning Corporation (Midland Mich.); Med-4805, Med-4810, Med-4820, Med-4830, Med-4840, Med-4850, Med-4860, Med-4870, Med-4880, Med-2245, Med-4515, Med-4516, Med-4715, Med-2174, Med-4535, Med-4550, Med-4565, Med-4719, Med-4720, Med-4725, Med-4735, Med-4756, Med-4765, Med-4780, Med-4755, Med-4770, Med-4211, Med-6210, Med-6233, or Med-6820 all from NuSil Technology (Carpinteria, Calif.). The hardness of the silicone, measured in durometers, may be in the range of 10 D to about 400 D, but is more likely in the range of 20 D to 300 D, and is preferably in the range of 25-200 D.

The elastomer may be treated for sterilization and/or to increase stability or durability of the device. For example, gamma radiation may be employed to sterilize the device before implantation; however, it has been observed that gamma radiation is also useful for cross-linking a silicone polymer. Cross-linking enhances the strength and stability of the elastomer, decreasing the number of particles that would be generated using an unradiated device. Other sterilization methods include heat treatment, autoclaving or gas sterilization such as with elthylene oxide. Other cross-linking methods or strengthening methods include storing the intervertebral disc of the present invention in a saline or other appripriate solution prior to implantation, chemical cross-linking methods such as by peroxide treatment, or heat treatment to enhance annealing. Gamma irradiation preferably is conducted in a vacuum or other neutral atmosphere so as to prevent oxidation.

### EXAMPLES

1. Materials and Methods:

Kinematic Text Fixture for Fatigue Testing. Fifth and sixth cervical vertebral bodies were obtained from a formalin-fixed cadaver and completely denuded of soft tissue. The height of the disc space between the vertebrae measured 6 mm. The caudal and cephalic surfaces of the C6 and C5 vertebral bodies respectively were mounted into the middle of two shallow cylindrical plastic containers by means of steel rods and polyurethane potting compound (4032-H, Vagabond Corp., Warmer Springs, Calif.). Two steel rods were used for each vertebral body. The steel rods were placed perpendicular to one another through the middle of the vertebral bodies and the walls of the container in the transverse plane. The container holding the C6 vertebral body was fixed to the top of a table. The container holding the C5 vertebral body was attached to a Panorobot (Panasonic, Japan) arm by means of a double-jointed steel rod. The two joints of the rod were perpendicular to one another. The two vertebral bodies were then anatomically aligned and the artificial disc was placed between the adjacent disc surfaces of the two vertebral bodies. Safety contact detectors were attached to each vertebral surface so that if the disc were extruded, the contact detector would stop the robot and a timing clock. Eight pounds of weight was place on the top of the container housing the fifth vertebral body (C5) to account for the weight of the head.

Single Unit Artificial Discs: Three types of single-unit artificial discs were tested: flat rectangular discs, recessed-edge discs and contoured discs. Two of the flat discs were made from implant grade HCRA 80 silicon (Applied Silicone Corp., Ventura, Calif.) with a durometer (hardness) of 85 (85 D), and four flat discs were made from implant grade A2189F silicon (Factor II, Inc., Lakeside, Ariz.) with a durometer of 30 (30 D). The flat discs were rectangular, measuring 12 mm in lateral distance and 11 mm in anterior-posterior distance, with a thickness of 6 mm.

Two recessed-edge discs were tested. The recessed-edge discs were the same in dimension as the flat discs, with the exception that 1 mm in thickness was removed along the length of either side of both the top and bottom surfaces of the device (the surfaces that are adjacent the vertebrae when the device is implanted) (see FIGS. 4A and 4B). The recessed-edge discs were made from implant grade HCRA 80 silicon with a durometer of 85 (85 D).

The contoured discs that were tested were formed using anaplasty technology from the molded contours of cadaver vertebrae. A wax blank was pressed between the distal vertebral plate of the C4 vertebra and the proximal vertebral plate of the C5 vertebra. Around this wax blank, a ceramic mold was poured, allowed to solidify, and the wax was then melted out. The mold was made in two parts, a top and a bottom. The two-part nature of the mold allowed the thickness to be adjusted for the next positive mold by placing spacers between the parts. From this negative ceramic mold, a couple of 6 mm-thick positive polyvinyl siloxane molds were made. The margin of the positive polyvinyl siloxane intervertebral device mold was trimmed so that the disc measured approximately 12 mm×12 mm. This positive mold was then used to make a second two-part negative mold that was then used to make the final custom intervertebral device. Silicone A2189F was used to make four implants of 30 durometer (30 D).

Fatigue Testing. The robot performed the following movements based on average human kinematic studies (see, e.g., Schulte, et al., Spine 14:1116-21 (1989)): flexion/extension: ±4.7°; lateral left/right bending: ±2.1°; and coupled rotation: ±3.8°. The following series of movements were performed for each cycle: [flexion/+rotation]; [extension/−rotation]; [zero position]; [lateral left/+rotation]; [lateral right/−rotation]; [zero position]; [flexion/−rotation]; [extension/+rotation]; [zero position]; [lateral left/−rotation]; [lateral right/+rotation]; [zero position]. One million cycles of the series of movements were performed on each disc.

Each disc was measured and weighed prior to each wear test and at the completion of the one million cycles. A micrometer was used to measure the width and length of each disc and its thickness. Thicknesses of the contoured discs were measured at three locations: the region that extends anteriorly beyond the vertebrae, the region between the anterior edges of the vertebrae and the region between the midsections of the vertebrae bodies.

Scanning electron microscopy was performed on the caudal and cepahic surfaces of one of the 30 D discs after the completion of one million cycles of movements, as well as on an unused, control 30 D specimen. In addition, particles were collected after a million cycles for both the 30 D and 85 D silicone formulations. Dynamic light scattering (DLSI, Brookhaven Instrument Corp.) was performed to analyze the size distribution of the particles under one μm in diameter.

2. Results:

The discs were stable in the disc space (see Table 1).

TABLE 1

| DISC TYPE | POSITION AFTER 1 MILLION CYCLES |
|---|---|
| Flat 85 (1) | stable |
| Flat 85 (2) | extruded |
| Flat 30 (1) | stable |
| Flat 30 (2) | stable |
| Flat 30 (3) | stable |
| Flat 30 (4) | stable |
| Recessed 85 (1) | stable |
| Recessed 85 (2) | stayed in place but rotated 90° |
| Contoured 30 (1) | stable |
| Contoured 30 (2) | stable |
| Contoured 30 (3) | stable |
| Contoured 30 (4) | stable |

None of the discs tested showed cracks or had breakage; however, one of the 85 D flat discs extruded after 0.2 million cycles. One recessed-edge disc of 85 D maintained its position in the disc space; however, the other recessed-edge disc stayed within the disc space but rotated around its central axis by 90 degrees. All contoured discs of 30 D maintained their position in the disc space.

Each disc was weighed after the completion of one million cycles. Both flat discs of 85 D showed weight loss; however, all flat discs of 30 D had an average weight gain of 8.3 mg. The recessed-edge discs each had a weight loss of between 5.0 and 6.0 mg. The contoured discs of 30 D showed an average increase in weight of approximately 8.3 mg. The weight gain that was observed in some of the discs was most probably due to absorption moisture from the atmosphere.

Dimensional changes for the discs generally were minimal. In the contoured disc group at the completion of one million cycles, decreases were observed in the thickness measurements of all three points averaging 0.2 mm, and increases were seen in the length and width of these discs.

One 30 D contoured disc was tested for 5 million cycles. After an initial weight gain of 12 mg at 2 million cycles, the disc showed a gradual loss of weight to 2.7 mg greater than the original weight. Four additional studies were conducted on flat discs with a hardness of 30 D. These discs were tested for 10 million cycles and showed no evidence of displacement.

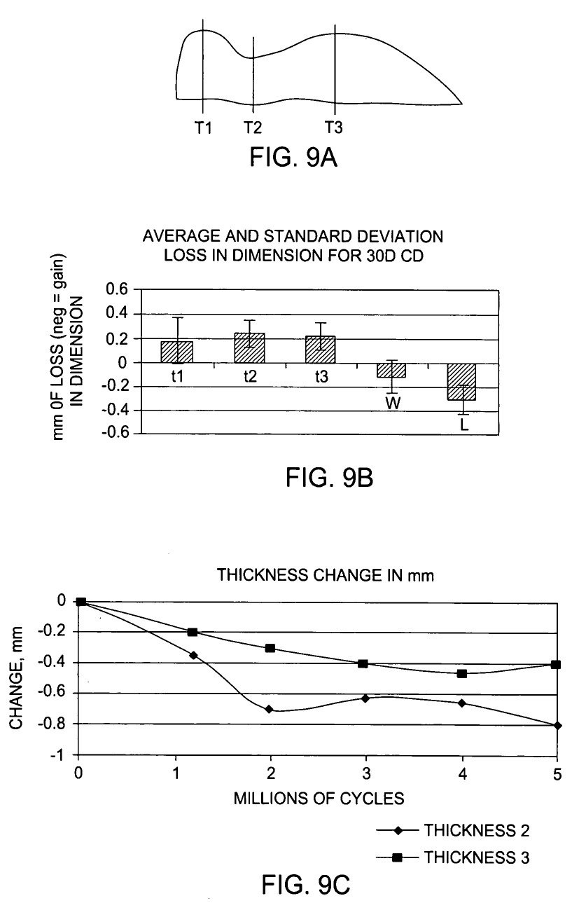

FIG. 9A is a side-sectional view of a contoured disc shown indicating the sites where thickness measurements were taken. FIG. 9B is a chart showing the average and standard deviation change in dimension in one 30 D contoured disc at one million cycles. The legends t1, t2, and t3 correspond to the positions shown at FIG. 9A, and w and l correspond to width and length, respectively. FIG. 9C is a chart showing the thickness change in mm for two sites directly between vertebrae for a 30 D contoured disc at five million cycles.

The results show that at the lower durometer (30 D), the flat, rectangular discs performed as well as the contoured discs. This allows for a truly simplified design, as the discs do not necessarily have to be custom designed. Also, at the lower durometer, weight change was minimal. The reason behind the overall lower wear for discs with a lower durometer is not known, but may be due to the increased pliability of the disc allowing for molding to the topography and dimensions of the space, thereby producing less friction between the disc and vertebral bodies during movement. Importantly, the results make clear that the intervertebral device of the present invention maintains its position in the disc space without an anchoring device, and that the discs are durable.

The wear rate and loss of thickness loss observed are well within the acceptable range for hip and knee implants. The decrease in thickness shown for the t2 and t3 dimension in the contoured implants appears to be largely due to creep of the material and not to wear. Compression in the thickness dimension would be expected to produce some expansion in the length and width dimension. Even in the dry unlubricated environment tested, the particles that were generated appear to be larger and fewer in numbers than those seen in polyethylene insert hip and knee implants; and thus would be expected to reduce greatly any toxic effects. The particles generated in polyethylene systems are almost entirely submicron particles—a size that has been found to be particularly toxic to macrophages and fibroblasts. Most of the particles generated by intervertebral device of the present invention were greater than 10 μm in diameter, a size that macrophages are not able to digest readily.

Scanning electron microscopy of particles collected from one 30 D contoured disc sample between 3 and 4 million cycles showed a wide range of particle sizes from under 1 μm up to 600 μm. The dynamic light scattering instrument (DLSI) was not able to give a meaningful particle size due to the diversity of particle sizes and the small number of particles in the sample.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material, or process to the objective, spirit and scope of the present invention. All such modifications are intended to be within the scope of the invention.

1. A prosthetic intervertebral device consisting essentially of a unitary body comprising an elastomer.

2. The prosthetic intervertebral device of claim 1, wherein the body is rectangular.

3. The prosthetic intervertebral device of claim 2, wherein the body has recessed edges along a width of upper and lower surfaces.

4. The prosthetic intervertebral device of claim 1, wherein the body is contoured.

5. The prosthetic intervertebral device of claim 1, wherein the body is disc shaped.

6. The prosthetic intervertebral device of claim 1, wherein the body ranges in width from about 6 mm to about 65 mm.

7. The prosthetic intervertebral device of claim 6, wherein the body ranges in width from about 8 mm to about 40 mm.

8. The prosthetic intervertebral device of claim 7, wherein the body ranges in width from about 10 mm to about 30 mm.

9. The prosthetic intervertebral device of claim 1, wherein the body ranges in length from about 8 mm to about 45 mm.

10. The prosthetic intervertebral device of claim 9, wherein the body ranges in length from about 10 mm to about 35 mm.

11. The prosthetic intervertebral device of claim 10, wherein the body ranges in length from about 12 mm to about 25 mm.

12. The prosthetic intervertebral device of claim 1, wherein the body ranges in thickness from about 2 mm to about 25 mm.

13. The prosthetic intervertebral device of claim 12, wherein the body ranges in thickness from about 3 mm to about 20 mm.

14. The prosthetic intervertebral device of claim 13, wherein the body ranges in thickness from about 5 mm to about 15 mm.

15. The prosthetic intervertebral device of claim 1, wherein the elastomer has a durometer of about 10 D to about 400 D.

16. The prosthetic intervertebral device of claim 15, wherein the elastomer has a durometer of about 20 D to about 300 D.

17. The prosthetic intervertebral device of claim 16, wherein the elastomer has a durometer of about 25 D to about 200 D.

18. The prosthetic intervertebral device of claim 1, wherein the elastomer comprises silicone.

19. A prosthetic intervertebral device consisting essentially of a unitary body consisting essentially of silicone with a durometer of about 25 D to about 200 D, wherein said body has a width of about 10 mm to about 30 mm, a length of about 10 mm to about 35 mm, and a thickness of about 5 mm to about 15 mm.

20. A method for preserving intervertebral disc space after discectomy comprising implanting the intervertebral device of claim 1 into the intervertebral disc space.
